(19) United States
(12) Patent Application Publication
(10) Pub. No.: US 2007/0149634 A1
(43) Pub. Date: Jun. 28, 2007

(54) Long chain polyether polyols

(76) Inventors: Karl W. Haider, Wexford, PA (US); Jose F. Pazos, Charleston, WV (US); Steven J. Rodberg, Charleston, WV (US); Daniel R. Wagner, Leverkusen (DE); Jack R. Reese II, Hurricane, WV (US); Barbara J. Buck, Cross Lanes, WV (US)

Correspondence Address: BAYER MATERIAL SCIENCE LLC, 100 BAYER ROAD, PITTSBURGH, PA, 15205, US

(21) Appl. No.: 11/315,667

(22) Filed: Dec. 22, 2005

(51) Int. Cl. C08G 18/00; C07C 43/11; C08F 20/00

(52) U.S. Cl. 521/172; 525/438; 568/679

(57) ABSTRACT

The present invention provides a long-chain polyether polyol having a number average molecular weight of greater than about 500 g/mole and produced by alkoxylating a polyoxyethylene-containing initiator with an alkylene oxide in the presence of a basic catalyst having at least one cation thereof chelated by the polyoxyethylene-containing initiator. The inventive long-chain polyether polyols may be used to provide flexible polyurethane foams and non-cellular polyurethanes.

### FIELD OF THE INVENTION

The present invention relates in general to polyether polyols, and more specifically, to a long-chain polyether polyol having a number average molecular weight of more than about 500 g/mole and produced by alkoxylating a polyoxyethylene-containing initiator with an alkylene oxide in the presence of a basic catalyst having at least one cation thereof chelated by the polyoxyethylene-containing initiator.

### BACKGROUND OF THE INVENTION

It has been known for many years that cyclic ethers complex potassium ions strongly. Crown ethers were discovered in the 1960's by Charles Pederson and in 1987 he was awarded the Nobel Prize for his efforts. The ability of cyclic ethers to strongly complex metal ions has led to much scientific work. Unfortunately, because crown ethers are difficult to make, expensive and highly toxic, they have never found wide commercial application. Perhaps because crown ethers were discovered first, many in the art have overlooked the strong complexing abilities possessed by non-cyclic polyethers. Among the advantages are the ready availability, low cost and the fact that polymers and oligomers of ethylene oxide are so non-toxic as to be acceptable for use as food additives.

The concept of using polyethylene glycols (“PEGs”) for rate enhancement of the KOH catalyzed alkoxylation of long-chain polyols is known in the art (See “Synthesis of Polyether Polyols for Flexible Polyurethane Foams with Complexed Counter-Ion” by Mihail Ionescu, Viorica Zugravu, Ioana Mihalache and Ion Vasile, Cellular Polymers IV, International Conference, 4th, Shrewsbury, UK, Jun. 5-6, 1997 Paper 8, 1-8. Editor(s): Buist, J. M.).

A commonly-assigned U.S. patent application filed on an even date herewith and entitled “Base-catalyzed alkoxylation in the presence of polyoxyethylene-containing compounds”, (Atty. Docket No. PO8708, U.S. Ser. No. ______) discloses a molecular weight dependency for a polyoxyethylene-containing additive which acts as a chelating agent in the base-catalyzed alkoxylation of long-chain polyethers.

A second commonly-assigned U.S. patent application also filed on an even date herewith and entitled “Base-catalyzed alkoxylation in the presence of non-linear polyoxyethylene-containing compounds”, (Atty. Docket No. PO8709, U.S. Ser. No. ______) discloses a non-linear, at least trifunctional polyoxyethylene-containing additive as a chelating agent for the base-catalyzed alkoxylation of long-chain polyethers, with no detrimental effect on flexible foams produced therefrom.

Finally, a third commonly-assigned U.S. patent application also filed on an even date herewith and entitled “Short chain polyether polyols for rigid polyurethane foam”, (Atty. Docket No. PO8707, U.S. Ser. No. ______) discloses a polyoxyethylene-containing additive as a chelating agent in the alkoxylation of short chain polyethers.

The present invention expands upon those teachings by using a polyoxyethylene-containing initiator to act as a chelating agent in the base-catalyzed production of long-chain polyether polyols, thus eliminating the need for the addition of a polyoxyethylene-containing additive.

### SUMMARY OF THE INVENTION

Accordingly, the present invention provides a long-chain polyether polyol having a number average molecular weight of more than about 500 g/mole and produced by alkoxylating a polyoxyethylene-containing initiator with an alkylene oxide in the presence of a basic catalyst having at least one cation thereof chelated by the polyoxyethylene-containing initiator. The inventive polyols may be used to provide flexible polyurethane foams and non-cellular polyurethanes.

These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages, OH numbers, functionalities and so forth in the specification are to be understood as being modified in all instances by the term “about.” Equivalent weights and molecular weights given herein are number average equivalent weights and number average molecular weights respectively, unless indicated otherwise.

The present invention provides a long-chain polyether polyol having a number average molecular weight of more than 500 g/mole and produced by alkoxylating a polyoxyethylene-containing initiator with an alkylene oxide in the presence of a basic catalyst having at least one cation thereof chelated by the polyoxyethylene-containing initiator.

The present invention further provides a process for producing a long-chain polyether polyol having a number average molecular weight of more than 500 g/mole involving alkoxylating a polyoxyethylene-containing initiator with an alkylene oxide in the presence of a basic catalyst having at least one cation thereof chelated by the polyoxyethylene-containing initiator.

The present invention yet further provides a polyurethane foam made from the reaction product of at least one polyisocyanate and a long-chain polyether polyol having a number average molecular weight of more than 500 g/mole and produced by alkoxylating a polyoxyethylene-containing initiator with an alkylene oxide in the presence of a basic catalyst having at least one cation thereof chelated by the polyoxyethylene-containing initiator, optionally in the presence of at least one of blowing agents, surfactants, other cross-linking agents, extending agents, pigments, flame retardants, catalysts and fillers.

The present invention still further provides a process for producing a polyurethane foam involving reacting at least one polyisocyanate with a long-chain polyether polyol having a number average molecular weight of more than 500 g/mole produced by alkoxylating a polyoxyethylene-containing initiator with an alkylene oxide in the presence of a basic catalyst having at least one cation thereof chelated by the polyoxyethylene-containing initiator, optionally in the presence of at least one of blowing agents, surfactants, other cross-linking agents, extending agents, pigments, flame retardants, catalysts and fillers.

By “long-chain” polyether polyol, the inventors herein mean a polyether polyol having a number average molecular weight of greater than 500 g/mole, preferably from 500 to 50,000 g/mole, more preferably from 1,000 to 30,000 g/mole, and most preferably from 1,000 to 8,000 g/mole. The molecular weight of the inventive long-chain polyether polyols may be in an amount ranging between any combination of these values, inclusive of the recited values.

The long-chain polyether polyols of the present invention are made by basic catalysis, the general conditions of which are familiar to those skilled in the art. The basic catalyst may be any basic catalyst known in the art, more preferably the basic catalyst is one of potassium hydroxide, sodium hydroxide, barium hydroxide and cesium hydroxide; most preferably the basic catalyst is potassium hydroxide.

The polyoxyethylene-containing initiators useful in the present invention are polyoxyethylene-containing polyether polyols having a molecular weight of less than 500 g/mole prepared by alkoxylating (with either ethylene oxide or mixtures of oxides containing ethylene oxide) any of the low molecular weight alcohols, amines, diols, diamines, polyols or polyamines known to those skilled in the art to be useful as starters for polyether polyols. These include, for example C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and the like.

The polyoxyethylene-containing starters useful in the present invention may preferably be produced at the same molecular weight as current starters used to prepare the polyols. Thus, the polyoxyethylene-containing accelerator is built directly into the initiator. This approach eliminates the need for the addition of a polyoxyethylene-containing additive prior to the alkoxylation as is taught in the three commonly assigned applications mentioned hereinabove. These initiators contain sufficient polyoxyethylene to result in the long-chain polyether polyol having a polyoxyethylene content of from 0.5 to 20 wt. %, more preferably from 1 to 10 wt. % and most preferably from 2 to 7 wt. %, based on the weight of the long-chain polyether. The polyoxyethylene-containing starter may be included in an amount such that the final polyoxyethylene content provided by the initiator ranges between any combination of these values, inclusive of the recited values.

The alkylene oxides useful in alkoxylating the initiator to produce the inventive long-chain polyether polyols include, but are not limited to, ethylene oxide, propylene oxide, oxetane, 1,2- and 2,3-butylene oxide, isobutylene oxide, epichlorohydrin, cyclohexene oxide, styrene oxide, and the higher alkylene oxides such as the C₅-C₃₀ α-alkylene oxides. Propylene oxide alone or mixtures of propylene oxide with ethylene oxide or another alkylene oxide are preferred. Other polymerizable monomers may be used as well, e.g. anhydrides and other monomers as disclosed in U.S. Pat. Nos. 3,404,109, 3,538,043 and 5,145,883, the contents of which are herein incorporated in their entireties by reference thereto.

The inventive long-chain polyether polyols may preferably be reacted with a polyisocyanate, optionally in the presence of one or more of blowing agents, surfactants, cross-linking agents, extending agents, pigments, flame retardants, catalysts and fillers to produce flexible polyurethane foams.

Suitable polyisocyanates are known to those skilled in the art and include unmodified isocyanates, modified polyisocyanates, and isocyanate prepolymers. Such organic polyisocyanates include aliphatic, cycloaliphatic, araliphatic, aromatic, and heterocyclic polyisocyanates of the type described, for example, by W. Siefken in Justus Liebigs Annalen der Chemie, 562, pages 75 to 136. Examples of such isocyanates include those represented by the formula
Q(NCO)ₙ
in which n is a number from 2-5, preferably 2-3, and Q is an aliphatic hydrocarbon group; a cycloaliphatic hydrocarbon group; an araliphatic hydrocarbon group; or an aromatic hydrocarbon group.

Examples of suitable isocyanates include ethylene diisocyanate; 1,4-tetramethylene diisocyanate; 1,6-hexamethylene diisocyanate; 1,12-dodecane diisocyanate; cyclobutane-1,3-diisocyanate; cyclohexane-1,3- and -1,4-diisocyanate, and mixtures of these isomers; 1-isocyanato-3,3,5-trimethyl-5-isocyanatomethylcyclohexane (isophorone diisocyanate. German Auslegeschrift 1,202,785 and U.S. Pat. No. 3,401,190); 2,4- and 2,6-hexahydrotoluene diisocyanate and mixtures of these isomers; dicyclohexylmethane-4,4′-diisocyanate (hydrogenated MDI, or HMDI); 1,3- and 1,4-phenylene diisocyanate; 2,4- and 2,6-toluene diisocyanate and mixtures of these isomers (TDI); diphenylmethane-2,4′- and/or -4,4′-diisocyanate (MDI); polymeric diphenylmethane diisocyanate (PMDI), naphthylene-1,5-diisocyanate; triphenylmethane-4,4′,4″-triisocyanate; polyphenyl-polymethylene-polyisocyanates of the type which may be obtained by condensing aniline with formaldehyde, followed by phosgenation (crude MDI), which are described, for example, in GB 878,430 and GB 848,671; norbornane diisocyanates, such as described in U.S. Pat. No. 3,492,330; m- and p-isocyanatophenyl sulfonylisocyanates of the type described in U.S. Pat. No. 3,454,606; perchlorinated aryl polyisocyanates of the type described, for example, in U.S. Pat. No. 3,227,138; modified polyisocyanates containing carbodiimide groups of the type described in U.S. Pat. No. 3,152,162; modified polyisocyanates containing urethane groups of the type described, for example, in U.S. Pat. Nos. 3,394,164 and 3,644,457; modified polyisocyanates containing allophanate groups of the type described, for example, in GB 994,890, BE 761,616, and NL 7,102,524; modified polyisocyanates containing isocyanurate groups of the type described, for example, in U.S. Pat. No. 3,002,973, German Patentschriften 1,022,789, 1,222,067 and 1,027,394, and German Offenlegungsschriften 1,919,034 and 2,004,048; modified polyisocyanates containing urea groups of the type described in German Patentschrift 1,230,778; polyisocyanates containing biuret groups of the type described, for example, in German Patentschrift 1,101,394, U.S. Pat. Nos. 3,124,605 and 3,201,372, and in GB 889,050; polyisocyanates obtained by telomerization reactions of the type described, for example, in U.S. Pat. No. 3,654,106; polyisocyanates containing ester groups of the type described, for example, in GB 965,474 and GB 1,072,956, in U.S. Pat. No. 3,567,763, and in German Patentschrift 1,231,688; reaction products of the above-mentioned isocyanates with acetals as described in German Patentschrift 1,072,385; and polyisocyanates containing polymeric fatty acid groups of the type described in U.S. Pat. No. 3,455,883. It is also possible to use the isocyanate-containing distillation residues accumulating in the production of isocyanates on a commercial scale, optionally in solution in one or more of the polyisocyanates mentioned above. Those skilled in the art will recognize that it is also possible to use mixtures of the polyisocyanates described above. Particularly preferred in the polyurethane foams of the present invention are 2,4- and 2,6-toluene diisocyanate and mixtures of these isomers (TDI).

Prepolymers may also be employed in the preparation of the inventive foams. Prepolymers may be prepared by reacting an excess of organic polyisocyanate or mixtures thereof with a minor amount of an active hydrogen-containing compound as determined by the well-known Zerewitinoff test, as described by Kohler in Journal of the American Chemical Society, 49, 3181(1927). These compounds and their methods of preparation are known to those skilled in the art. The use of any one specific active hydrogen compound is not critical; any such compound can be employed in the practice of the present invention.

Suitable additives optionally included in the polyurethane forming formulations of the present invention include, for example, stabilizers, catalysts, cell regulators, reaction inhibitors, plasticizers, fillers, crosslinking or extending agents, blowing agents, etc.

Stabilizers which may be considered suitable for the inventive foam forming process include, for example, polyether siloxanes, and preferably those which are insoluble in water. Compounds such as these are generally of such a structure that a relatively short chain copolymer of ethylene oxide and propylene oxide is attached to a polydimethylsiloxane residue. Such stabilizers are described in, for example, U.S. Pat. Nos. 2,834,748, 2,917,480 and 3,629,308.

Catalysts suitable for the foam forming process of the present invention include those which are known in the art. These catalysts include, for example, tertiary amines, such as triethylamine, tributylamine, N-methylmorpholine, N-ethylmorpholine, N,N,N′,N′-tetramethylethylenediamine, pentamethyl-diethylenetriamine and higher homologues (as described in, for example, DE-A 2,624,527 and 2,624,528), 1,4-diazabicyclo(2.2.2)octane, N-methyl-N′-dimethyl-aminoethylpiperazine, bis-(dimethylaminoalkyl)piperazines, N,N-dimethylbenzylamine, N,N-dimethylcyclohexylamine, N,N-diethyl-benzylamine, bis-(N,N-diethylaminoethyl)adipate, N,N,N′,N′-tetramethyl-1,3-butanediamine, N,N-dimethyl-β-phenylethylamine, 1,2-dimethylimidazole, 2-methylimidazole, monocyclic and bicyclic amines together with bis-(dialkylamino)alkyl ethers, such as 2,2-bis-(dimethylaminoethyl)ether.

Other suitable catalysts which may be used in producing the inventive polyurethane foams include, for example, organometallic compounds, and particularly, organotin compounds. Organotin compounds which may be considered suitable include those organotin compounds containing sulfur. Such catalysts include, for example, di-n-octyltin mercaptide. Other types of suitable organotin catalysts include, preferably tin(II) salts of carboxylic acids such as, for example, tin(II) acetate, tin(II) octoate, tin(II) ethylhexoate and/or tin(II) laurate, and tin(IV) compounds such as, for example, dibutyltin oxide, dibutyltin dichloride, dibutyltin diacetate, dibutyltin dilaurate, dibutyltin maleate and/or dioctyltin diacetate.

Water is preferably used as the sole blowing agent in the foams made according to the present invention, although auxiliary blowing agents, such as, for example, carbon dioxide, can be used. Water functions as the blowing by reacting with the isocyanate component to chemically form carbon dioxide gas plus an amine moiety which reacts further with the polyisocyanate to form urea backbone groups.

Further examples of suitable additives, which may optionally be included in the flexible polyurethane foams of the present invention can be found in Kunststoff-Handbuch, volume VII, edited by Vieweg & Hochtlen, Carl Hanser Verlag, Munich 1993, 3^{rd} Ed., pp. 104 to 127, for example. The relevant details concerning the use and mode of action of these additives are set forth therein.

### EXAMPLES

The present invention is further illustrated, but is not to be limited, by the following examples. All quantities given in “parts” and “percents” are understood to be by weight, unless otherwise indicated. The following materials were used in the Examples:
Polyol A: a polyether polyol initiator based on propoxylated glycerine having a hydroxyl number of 350 mg KOH/g, contains 4 wt. % KOH;
Polyol B: a polyether polyol initiator based on propoxylated sorbitol having a hydroxyl number of 200 mg KOH/g, contains 2.2 wt. % KOH;
Polyol C: a polyoxyethylene-containing polyether polyol initiator having a hydroxyl number of ˜350 mg KOH/g, prepared by ethoxylating glycerine with approximately 8.8 moles of ethylene oxide per mole of glycerine, contains 4 wt. % KOH;
Polyol D: a polyoxyethylene-containing polyether polyol initiator having a hydroxyl number of ˜350 mg KOH/g, prepared by ethoxylating glycerine first with ethylene oxide (˜4.4 moles ethylene oxide per mole of glycerine and subsequently with ˜3.4 moles of propylene oxide per mole of glycerine, contains 4 wt. % KOH.

The inventive concept was applied to the synthesis of an ethylene oxide-capped molded foam triol (a glycerin-sorbitol based polyether having a hydroxyl number of about 31.5 mg KOH/g that has 16% ethylene oxide cap).

### Example C-1

In this comparative example, a start mixture having a hydroxyl number of 290 mg KOH/g was prepared from 60% Polyol A (120 g) and 40% Polyol B (80 g). This mixture was charged to a one-liter polyether polyol reactor and propoxylated in two stages to a final hydroxyl number of 37 mg KOH/g. In the first stage, the 200 g of start mixture were heated under vacuum (˜0.5 psia) at 105° C., while allowing nitrogen to flow through the reactor. After thirty minutes, the nitrogen feed was stopped, and the vacuum valve was closed, thus blocking the vacuum in the reactor. Propylene oxide (400 g) was fed into the reactor at a rate sufficient to maintain 40 psia reactor pressure. The time required to complete the 400 g feed was measured and used to calculate a feed rate (g/min.) for the first stage of the propoxylation.

The reaction mixture was allowed to continue stirring at 105° C., until the propylene oxide was consumed, as evidenced by the pressure reaching a steady state value. The contents of the reactor were withdrawn, and 200 g of this product was added back into the reactor. In the second stage, the time required to feed 322 g of propylene oxide to this material under the same conditions of temperature and pressure as detailed above, thus decreasing the hydroxyl number from 97 to 37 mg KOH/g, was determined and similarly used to determine the oxide feed rate.

### Examples 2 and 3

The long-chain polyethers of Examples 2 and 3 were made according to the procedure given above for Example C-1, except that Polyol A of the start mixture was replaced either by Polyol C (Example 2) or Polyol D (Example 3).

The propoxylation rate of start mixtures containing each of these polyoxyethylene-containing starters were compared with that for the standard starter mixture (Ex. C-1). The propoxylation rate was determined at 105° C. for both stages of the propoxylation Polyoxyethylene content in each sample at the end of each stage, along with propoxylation rate during each of the two stages of the alkoxylation are summarized below in Table I.

As can be appreciated by reference to Table I, the polyoxyethylene-containing starters, Polyol C (Ex. 2) and Polyol D (Ex. 3) gave higher propoxylation rates than the control in the first portion of the alkoxylation. In the most dramatic example, the propoxylation rate increased from 2.39 g/min. (Ex. C-1) to 3.57 g/min. for the long-chain polyether polyol made in Example 2 from Polyol C (14.7% total polyoxyethylene content at end of this feed). At lower levels of ethylene oxide in the starter in Example 3 using Polyol D (approximately 7% polyoxyethylene content at the end of this feed), the feed rate of 3.03 g/min. was still markedly higher than the 2.39 g/min. rate of the control.

### Examples C4, C5, 6, and 7

The polyoxyethylene-containing polyether polyol initiators (Polyols C and D) were evaluated as starters on a larger scale. In a five-gallon polyether polyol reactor, a start mixture was prepared from 60% Polyol A and 40% Polyol B. This start mixture (hydroxyl number of 290 mg KOH/g) was stripped under vacuum (˜0.5 psia) at 105° C., while allowing nitrogen to flow through the reactor. After thirty minutes, the nitrogen feed was stopped, and the vacuum valve was closed, thus blocking the vacuum in the reactor. The mixture was propoxylated at 105° C. in a single stage to a final hydroxyl number of 37 mg KOH/g. The propylene oxide was fed at a constant rate sufficient to give either a seven-hour feed (Example C-4) or five-hour feed Example C-5). During the propoxylation, the reactor pressure was monitored, and the peak pressure was recorded.

Following the propoxylation, the polyols were ethoxylated in a second step (117° C.) to a theoretical hydroxyl number of 31.5 mg KOH/g. An analogous procedure was used to prepare long-chain polyether polyols of the present invention using a five-hour feed time, in which Polyol A in the start mixture was replaced either by Polyol C (Ex. 6) or Polyol D (Ex. 7). The pressure observed was indicative of the free propylene oxide concentration during the propoxylation, with a lower pressure at the same feed time corresponding to lower propylene oxide concentration and indicating higher reactivity. The polyoxyethylene-containing starters (Polyols C and D), at five hour feed times, gave pressures between those observed in the five (Ex. C-5) and seven (Ex. C-4) hour controls, indicating a greater reactivity than the comparative examples, which had no polyoxyethylene-containing initiators in the start mixture. Following the propoxylation, the long-chain polyols were ethoxylated in a procedure analogous to that used for C-4 and C-5 to a hydroxyl number of 31.5 mg KOH/g.

The physical properties for each of the polyols are summarized in Table II. There was no indication that the long-chain polyether polyols prepared using the polyoxyethylene-containing starters had a property profile which would negatively influence their utility in molded polyurethane foams.

TABLE I

| | | Polyoxyethylene | PO feed rate from | Relative PO | Relative PO |
|---|---|---|---|---|---|
| Ex. | | content (wt. %) at | 290 OH to 97 OH | Feed rate from | Feed rate from |
| No. | Starter Description | 97 and 37 OH no. | (g/min.) | 290 OH to 97 OH | 97 OH to 37 OH |
| C-1 | 60% Polyol A:40% Polyol B | 0; 0 | 2.39 | 1.00 | 1.00 |
| 2 | 60% Polyol C:40% Polyol B | 14.7; 5.6 | 3.57 | 1.49 | 1.02 |
| 3 | 60% Polyol D:40% Polyol B | 7.4; 2.8 | 3.03 | 1.27 | 1.01 |

TABLE II

| Ex. | | Feed Time | OH no. | Max. Press. | Viscosity | Unsaturation | Cloud Point |
|---|---|---|---|---|---|---|---|
| No. | Description | (hr.) | (Exp.) | (psia) | (cks) | (meq/g) | (° C.) |
| C-4 | 60% Polyol A:40% Polyol B | 7 | 33.4 | 57 | 1043 | 0.059 | 53.6 |
| C-5 | 60% Polyol A:40% Polyol B | 5 | 40.1 | 70 | 847 | 0.047 | 52.3 |
| 6 | 60% Polyol C:40% Polyol B | 5 | 32.9 | 65 | 984 | 0.045 | 55 |
| 7 | 60% Polyol D:40% Polyol B | 5 | 35.1 | 62 | 825 | 0.042 | 52.8 |

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

1. A long-chain polyether polyol having a number average molecular weight of greater than about 500 g/mole and produced by alkoxylating a polyoxyethylene-containing initiator with an alkylene oxide in the presence of a basic catalyst having at least one cation thereof chelated by the polyoxyethylene-containing initiator.

2. The long-chain polyether polyol according to claim 1 having a polyoxyethylene content of at least about 0.5 wt. %, based on the weight of the long-chain polyether polyol, which is provided by the polyoxyethylene-containing initiator.

3. The long-chain polyether polyol according to claim 1 having a polyoxyethylene content of from about 1 wt. % to about 10 wt. %, based on the weight of the long-chain polyether polyol, which is provided by the polyoxyethylene-containing initiator.

4. The long-chain polyether polyol according to claim 1 having a polyoxyethylene content of from about 2 wt. % to about 7 wt. %, based on the weight of the long-chain polyether polyol, which is provided by the polyoxyethylene-containing initiator.

5. The long-chain polyether polyol according to claim 1, wherein the starter for producing the polyoxyethylene-containing initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

6. The long-chain polyether polyol according to claim 1, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

7. The long-chain polyether polyol according to claim 1, wherein the alkylene oxide is propylene oxide or a block of propylene oxide followed by a block of ethylene oxide.

8. The long-chain polyether polyol according to claim 1, wherein the basic catalyst is chosen from potassium hydroxide, sodium hydroxide, barium hydroxide and cesium hydroxide.

9. The long-chain polyether polyol according to claim 1, wherein the basic catalyst is potassium hydroxide.

10. The long-chain polyether polyol according to claim 1 having a number average molecular weight of from about 500 g/mole to about 50,000 g/mole.

11. The long-chain polyether polyol according to claim 1 having a number average molecular weight of from about 1,000 g/mole to about 30,000 g/mole.

12. The long-chain polyether polyol according to claim 1 having a number average molecular weight of from about 1,000 g/mole to about 8,000 g/mole.

13. A process for producing a long-chain polyether polyol having a number average molecular weight of greater than about 500 g/mole comprising alkoxylating a polyoxyethylene-containing initiator with an alkylene oxide in the presence of a basic catalyst having at least one cation thereof chelated by the polyoxyethylene-containing initiator.

14. The process according to claim 13, wherein the long-chain polyether polyol has a polyoxyethylene content of at least about 0.5 wt. %, based on the weight of the long-chain polyether polyol, which is provided by the polyoxyethylene-containing initiator.

15. The process according to claim 13, wherein the long-chain polyether polyol has a polyoxyethylene content of from about 1 wt. % to about 10 wt. %, based on the weight of the long-chain polyether polyol, which is provided by the polyoxyethylene-containing initiator.

16. The process according to claim 13, wherein the long-chain polyether polyol has a polyoxyethylene content of from about 2 wt. % to about 7 wt. %, based on the weight of the long-chain polyether polyol, which is provided by the polyoxyethylene-containing initiator.

17. The process according to claim 13, wherein the starter for producing the polyoxyethylene-containing initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

18. The process according to claim 13, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

19. The process according to claim 13, wherein the alkylene oxide is propylene oxide or a block of propylene oxide followed by a block of ethylene oxide.

20. The process according to claim 13, wherein the basic catalyst is chosen from potassium hydroxide, sodium hydroxide, barium hydroxide and cesium hydroxide.

21. The process according to claim 13, wherein the basic catalyst is potassium hydroxide.

22. The process according to claim 13, wherein the long-chain polyether polyol has a number average molecular weight of from about 500 g/mole to about 50,000 g/mole.

23. The process according to claim 13, wherein the long-chain polyether polyol has a number average molecular weight of from about 1,000 g/mole to about 30,000 g/mole.

24. The process according to claim 13, wherein the long-chain polyether polyol has a number average molecular weight of from about 1,000 g/mole to about 8,000 g/mole.

25. A flexible polyurethane foam comprising the reaction product of at least one polyisocyanate; and
a long-chain polyether polyol having a number average molecular weight of greater than about 500 g/mole and produced by alkoxylating a polyoxyethylene-containing initiator with an alkylene oxide in the presence of a basic catalyst having at least one cation thereof chelated by the polyoxyethylene-containing initiator,
optionally in the presence of at least one of blowing agents, surfactants, other cross-linking agents, extending agents, pigments, flame retardants, catalysts and fillers.

26. The flexible polyurethane foam according to claim 23, wherein the at least one polyisocyanate is chosen from ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3-and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diisocyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-terminated prepolymers and mixtures thereof.

27. The flexible polyurethane foam according to claim 25, wherein the at least one polyisocyanate is chosen from 2,4- and 2,6-toluene diisocyanate and mixtures thereof (TDI).

28. The flexible polyurethane foam according to claim 25, wherein the long-chain polyether polyol has a polyoxyethylene content of at least about 0.5 wt. %, based on the weight of the long-chain polyether polyol, which is provided by the polyoxyethylene-containing initiator.

29. The flexible polyurethane foam according to claim 25, wherein the long-chain polyether polyol has a polyoxyethylene content of from about 1 wt. % to about 10 wt. %, based on the weight of the long-chain polyether polyol, which is provided by the polyoxyethylene-containing initiator.

30. The flexible polyurethane foam according to claim 25, wherein the long-chain polyether polyol has a polyoxyethylene content of from about 2 wt. % to about 7 wt. %, based on the weight of the long-chain polyether polyol, which is provided by the polyoxyethylene-containing initiator.

31. The flexible polyurethane foam according to claim 25, wherein the starter for producing the polyoxyethylene-containing initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

32. The flexible polyurethane foam according to claim 25, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

33. The flexible polyurethane foam according to claim 25, wherein the alkylene oxide is propylene oxide or a block of propylene oxide followed by a block of ethylene oxide.

34. The flexible polyurethane foam according to claim 25, wherein the basic catalyst is chosen from potassium hydroxide, sodium hydroxide, barium hydroxide and cesium hydroxide.

35. The flexible polyurethane foam according to claim 25, wherein the basic catalyst is potassium hydroxide.

36. The flexible polyurethane foam according to claim 25, wherein the long-chain polyether polyol has a number average molecular weight of from about 500 g/mole to about 50,000 g/mole.

37. The flexible polyurethane foam according to claim 25, wherein the long-chain polyether polyol has a number average molecular weight of from about 1,000 g/mole to about 30,000 g/mole.

38. The flexible polyurethane foam according to claim 25, wherein the long-chain polyether polyol has a number average molecular weight of from about 1,000 g/mole to about 8,000 g/mole.

39. A process for producing a flexible polyurethane foam comprising reacting
at least one polyisocyanate; with
a long-chain polyether polyol having a number average molecular weight of greater than about 500 g/mole and produced by alkoxylating a polyoxyethylene-containing initiator with an alkylene oxide in the presence of a basic catalyst having at least one cation thereof chelated by the polyoxyethylene-containing initiator,
optionally in the presence of at least one of blowing agents, surfactants, other cross-linking agents, extending agents, pigments, flame retardants, catalysts and fillers.

40. The process according to claim 39, wherein the at least one polyisocyanate is chosen from ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3-and -1,4-diisocyanate, 1-isocyanato-3,3,5-trimethyl-5-isocyanatomethyl-cyclohexane (isophorone diisocyanate), 2,4- and 2,6-hexahydrotoluene diisocyanate, dicyclohexylmethane-4,4′-diisocyanate (hydrogenated MDI, or HMDI), 1,3- and 1,4-phenylene diisocyanate, 2,4- and 2,6-toluene diisocyanate (TDI), diphenylmethane-2,4′- and/or -4,4′-diisocyanate (MDI), polymeric diphenylmethane diisocyanate (PMDI), naphthylene-1,5-diisocyanate, triphenyl-methane-4,4′,4″-triisocyanate, polyphenyl-polymethylene-polyisocyanates (crude MDI), norbornane diisocyanates, m- and p-isocyanatophenyl sulfonylisocyanates, perchlorinated aryl polyisocyanates, carbodiimide-modified polyisocyanates, urethane-modified polyisocyanates, allophanate-modified polyisocyanates, isocyanurate-modified polyisocyanates, urea-modified polyisocyanates, biuret containing polyisocyanates, isocyanate-term inated prepolymers and mixtures thereof.

41. The process according to claim 39, wherein the at least one polyisocyanate is chosen from 2,4- and 2,6-toluene diisocyanate and mixtures thereof (TDI).

42. The process according to claim 39, wherein the long-chain polyether polyol has a polyoxyethylene content of at least about 0.5 wt. %, based on the weight of the long-chain polyether polyol, which is provided by the polyoxyethylene-containing initiator.

43. The process according to claim 39, wherein the long-chain polyether polyol has a polyoxyethylene content of from about 1 wt. % to about 10 wt. %, based on the weight of the long-chain polyether polyol, which is provided by the polyoxyethylene-containing initiator.

44. The process according to claim 39, wherein the long-chain polyether polyol has a polyoxyethylene content of from about 2 wt. % to about 7 wt. %, based on the weight of the long-chain polyether polyol, which is provided by the polyoxyethylene-containing initiator.

45. The process according to claim 39, wherein the starter for producing the polyoxyethylene-containing initiator is chosen from C₁-C₃₀ monols, ethylene glycol, diethylene glycol, triethylene glycol, propylene glycol, 1,3-propanediol, dipropylene glycol, tripropylene glycol, neopentyl glycol, 1,4-butanediol, 1,2-butanediol, 2,3-butanediol, 1,3-butanediol, 1,6-hexanediol, glycerin, trimethylolpropane, trimethylolethane, pentaerythritol, α-methylglucoside, sorbitol, mannitol, hydroxymethylglucoside, hydroxypropylglucoside, sucrose, N,N,N′,N′-tetrakis[2-hydroxyethyl or 2-hydroxypropyl]ethylene diamine, 1,4-cyclohexanediol, cyclohexanedimethanol, hydroquinone, resorcinol, and mixtures thereof.

46. The process according to claim 39, wherein the alkylene oxide is chosen from ethylene oxide, propylene oxide, oxetane, 1,2- and 2,3-butylene oxide, isobutylene oxide, epichlorohydrin, cyclohexene oxide, styrene oxide, C₅-C₃₀ α-alkylene oxides and mixtures thereof.

47. The process according to claim 39, wherein the alkylene oxide is propylene oxide or a block of propylene oxide followed by a block of ethylene oxide.

48. The process according to claim 39, wherein the basic catalyst is chosen from potassium hydroxide, sodium hydroxide, barium hydroxide and cesium hydroxide.

49. The process according to claim 39, wherein the basic catalyst is potassium hydroxide.

50. The process according to claim 39, wherein the long-chain polyether polyol has a number average molecular weight of from about 500 g/mole to about 50,000 g/mole.

51. The process according to claim 39, wherein the long-chain polyether polyol has a number average molecular weight of from about 1,000 g/mole to about 30,000 g/mole.

52. The process according to claim 39, wherein the long-chain polyether polyol has a number average molecular weight of from about 1,000 g/mole to about 8,000 g/mole.
